(19) United States
(12) Patent Application Publication
(10) Pub. No.: US 2008/0103179 A1
(43) Pub. Date: May 1, 2008

(54) Combination Therapy

(76) Inventors: Peter Y. Tam, Redwood City, CA (US); Leland F. Wilson, Menlo Park, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(21) Appl. No.: 11/764,116

(22) Filed: Jun. 15, 2007

Related U.S. Application Data
(60) Provisional application No. 60/854,756, filed on Oct. 27, 2006.

(51) Int. Cl. A61K 31/133; A61K 31/135; A61K 31/35; A61K 31/423; A61K 31/435; A61P 25/00; A61P 25/08; A61P 25/32; A61P 3/10; A61P 43/00; A61P 9/12

(52) U.S. Cl. 514/326; 514/379; 514/454; 514/649; 514/653; 514/789

(57) ABSTRACT

The present invention features a novel therapy for treating diabetes, hypertension, migraine, epilepsy, sleep apnea, depression, impulse control disorders or alcohol addiction which involves treating a subject with a sympathomimetic agent (e.g., phentermine or a phentermine-like drug) in combination with an anticonvulsant sulfamate compound (e.g., topiramate) or an anticonvulsive sulfonylurea compound (e.g. zonisamide).

### CROSS-REFERENCE TO RELATED APPLICATIONS

This application claims priority under 35 U.S.C. §119(e)(1) to Provisional U.S. Patent Applications Ser. No. 60/854,756, filed Oct. 27, 2006. The disclosures of the aforementioned applications are incorporated by reference in their entireties.

### FIELD OF THE INVENTION

This invention relates to compositions and methods for treatment of diabetes, hypertension, migraine, epilepsy, sleep apnea, depression, impulse control disorders or alcohol addiction comprising antiepileptic agent and a sympathomimetic agent.

### BACKGROUND OF THE INVENTION

### Diabetes

Diabetes mellitus is associated with continuous and pathologically elevated blood glucose concentration. It is one of the leading causes of death in the United States and is responsible for about 5% of all mortality. Diabetes is divided into two major sub-classes-Type I, also known as juvenile diabetes, or Insulin-Dependent Diabetes Mellitus (IDDM), and Type II, also known as adult onset diabetes, or Non-Insulin-Dependent Diabetes Mellitus (NIDDM).

According to the American Diabetes Association, there are over one million juvenile diabetics in the United States. Type I Diabetes is a form of autoimmune disease. Autoantibodies produced by the patients completely or partially destroy the insulin producing cells of the pancreas. Juvenile diabetics must, therefore, receive exogenous insulin during their lifetime. Without treatment, excessive acidosis, dehydration, kidney damage, and death may result. Even with treatment, complications such as blindness, atherosclerosis, and impotence can occur.

There are more than five million Type II (adult onset) diabetics diagnosed in the United States. Type II disease usually begins during middle age; the exact cause is unknown. In Type II diabetics, rising blood glucose levels after meals do not properly stimulate insulin production by the pancreas. Additionally, peripheral tissues are generally resistant to the effects of insulin. The resulting high blood glucose levels (hyperglycemia) can cause extensive tissue damage. Type II diabetics are often referred to as insulin resistant. They often have higher than normal plasma insulin levels (hyperinsulinemia) as the body attempts to overcome its insulin resistance. Some researchers now believe that hyperinsulinemia may be a causative factor in the development of high blood pressure, high levels of circulating low density lipo-proteins (LDLs), and lower than normal levels of the beneficial high density lipo-proteins (HDLs). While moderate insulin resistance can be compensated for in the early stages of Type II diabetes by increased insulin secretion, in advanced disease states insulin secretion is also impaired.

Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. According to the World Health Organization, approximately 11% of the U.S. population between the ages of 20 and 74 are estimated to have impaired glucose tolerance. These individuals are considered to be at higher risk for diabetes and coronary artery disease.

Obesity may also be associated with insulin resistance. A causal linkage among obesity, impaired glucose tolerance, and Type II diabetes has been proposed, but a physiological basis has not yet been established. Some researchers believe that impaired glucose tolerance and diabetes are clinically observed and diagnosed only later in the disease process after a person has developed insulin resistance and hyperinsulinemia.

Insulin resistance is frequently associated with hypertension, coronary artery disease (arteriosclerosis), and lactic acidosis, as well as related disease states. The fundamental relationship between these disease states, and a method of treatment, has not been established.

### Hypertension

Hypertension is a condition that occurs when the blood pressure inside the large arteries is too high. Hypertension is very common, affecting about 50 million people in the United States alone. It is more common as people grow older and is both more common and more serious in African Americans. Most cases of hypertension are of unknown etiology, It is known that the tendency to develop hypertension can be inherited. Environment also plays a very important role in hypertension. For example, hypertension may be avoided by keeping body weight under control, keeping physically fit, eating a healthy diet, limiting alcohol intake, and avoiding medications that might increase blood pressure. Other less common causes of hypertension include disorders of the kidneys or endocrine glands. Hypertension has been called “the silent killer” because it has no specific symptoms and yet can lead to death. People with untreated hypertension are much more likely to die from or be disabled by cardiovascular complications such as strokes, heart attacks, heart failure, heart rhythm irregularities, and kidney failure, than people who have normal blood pressure.

Current treatments for hypertension include lifestyle changes (diet, exercise, nonsmoking, etc.) as well as drug therapy. The major classes of medications currently used to treat hypertension include adrenergic neuron antagonists (which are peripherally acting), alpha adrenergic agonists (which are centrally acting), alpha adrenergic blockers, alpha and beta blockers, angiotensin II receptor blockers, angiotensin converting enzyme (ACE) inhibitors, beta adrenergic blockers, calcium channel blockers, Thiazide and related diuretics, and vasodilators (which act by direct relaxation of vascular smooth muscles).

A particularly serious hypertensive disorder is primary pulmonary hypertension, also known as idiopathic pulmonary hypertension. This is a condition in which the blood pressure in the pulmonary arteries is abnormally high in the absence of other diseases of the heart or lungs. The cause of primary pulmonary hypertension is unknown. Pulmonary hypertension develops in response to increased resistance to blood flow. Narrowing of the pulmonary arterioles occurs and the right side of the heart becomes enlarged due to the increased work of pumping blood against the resistance. Eventually, progressive heart failure develops. Currently, there is no known cure for primary pulmonary hypertension. Treatment is primarily directed towards controlling the symptoms, although some success has occurred with the use of vasodilators. Other medications used to treat the symptoms of primary pulmonary hypertension include diuretics and calcium channel blockers. Typically, as the disease progresses, oxygen is often required. In certain cases, a heart-lung transplant may be indicated for certain suitable candidates, although the availability of donor organs continues to be extremely limited. Unfortunately, primary pulmonary hypertension is a progressive disease, usually leading to congestive heart failure and respiratory failure.

Secondary pulmonary hypertension is a serious disorder that arises as a complication of other conditions such as, for example, scleroderrna. Treatments are similar as those for primary pulmonary hypertension and, unfortunately, the prognosis is the same as well.

### Epilepsy

Epilepsy is defined as a brain disorder with recurrent, unprovoked seizures. Epileptic seizures are the clinically observed manifestations of excessive and/or hypersynchronous abnormal activity of neurons in the cerebral cortex. Nonepileptic seizures are related to a provoked event, such as metabolic derangement; toxins, such as cocaine; withdrawal from a particular drug, such as post alcoholic binge drinking, or acute head trauma. The behavioral features of a seizure reflect function of the portion of the cortex where the hyperactivity is occurring. Seizures can be generalized and appear to involve the entire brain simultaneously. Generalized seizures that result in only the loss of awareness are called absence seizures (previously referred to as “petit mal”). Alternatively, the generalized seizure may result in a convulsion with tonic-clonic contractions of the muscles (“grand mal” seizure). Some types of seizures, partial seizures, begin in one part of the brain and therefore start locally, but then may generalize. Focal seizures may generalize so rapidly that the seizure appears generalized from the onset. If the person remains somewhat conscious appearing and the seizure does not generalize then the seizure is called a complex partial seizure. Epilepsy is a common disorder with multiple etiologies that either produce brain damage or a cellular metabolic disturbance intrinsic to neurons. Epilepsy occurs in 0.5 to 1.0% of the population.

The most common treatment for epilepsy is drug therapy. Anti-epileptic agents effectively treat 50% of patients with epilepsy. The remaining patients either have recurrent seizures or have side effects. In the event that drug therapy proves unsuccessful, there are a number of other more invasive therapies that have shown improvements in seizure frequency. These other therapies typically involve surgery. The treatment may be electrical stimulation of the brain such as thalamic stimulation (Sussman et al. Epilepsia 29; 677; 1988), electrical stimulation of the peripheral nervous system such as vagal nerve stimulation, removal of the focal epileptic tissue such as temporal lobectomy (Barry et at. Archives of Neurology 49;21-27; 1992) or focal topectomy or by severing axonal pathways such as corpus callosotomy (Sussman N M et al. Neurology 37;350-354; 1987). There still remains a substantial number of patients whose epilepsy treatment does not provide adequate results.

### Migraines

There are two major types of migraines. The common migraine affects 80-85% of migraine sufferers and classical migraine with aura affects 15% of migraine sufferers. The common migraine is typically associated with various psychological (e.g., irritability, depression, fatigue, drowsiness, and restlessness), neurological (e.g., photophobia, and phonophobia), and gastrointestinal symptoms. The headache starts with mild pain, which increases in intensity over a short period of time. In some cases, early management of the headache can reduce the duration and severity of the pain. Headaches in classical migraines are typically characterized by a neurological deficit known as an aura. Exemplary deficits include visual scotoma or visual designs, hemiplegia, migrating paraesthesia, dysarthria, dysphasia, and deja-vus. The headache is usually accompanied by light or sound sensitivity, photophobia or phonophobia, irritability and impaired concentration. Treatment of the classical migraine at the time of the aura may alleviate the severity and duration of the headache.

Currently available drugs to alleviate the pain associated with migraines have modest or limited efficacy and are associated with various debilitating side effects. Thus, better therapies are needed for the management of migraines.

### Sleep Apnea

Sleep apnea occurs in two main types: obstructive sleep apnea, the more common form that occurs when throat muscles relax, and central sleep apnea, which occurs when your brain doesn't send proper signals to the muscles that control breathing. Additionally, some people have mixed sleep apnea, which is a combination of both obstructive and central sleep apneas. Sleep apnea means “cessation of breath.” It is characterized by repetitive episodes of upper airway obstruction that occur during sleep, usually associated with a reduction in blood oxygen saturation. In other words, the airway becomes obstructed at several possible sites. The upper airway can be obstructed by excess tissue in the airway, large tonsils, a large tongue and usually includes the airway muscles relaxing and collapsing when asleep. Another site of obstruction can be the nasal passages. Sometimes the structure of the jaw and airway can be a factor in sleep apnea.

The signs and symptoms of obstructive and central sleep apneas overlap, sometimes making the type of sleep apnea more difficult to determine. The most common signs and symptoms of obstructive and central sleep apneas include: excessive daytime sleepiness (hypersomnia); loud snoring; observed episodes of breathing cessation during sleep; abrupt awakenings accompanied by shortness of breath; awakening with a dry mouth or sore throat; morning headache; and/or difficulty staying asleep (insomnia). Disruptive snoring may be a more prominent characteristic of obstructive sleep apnea, while awakening with shortness of breath may be more common with central sleep apnea.

Sleep apnea is a progressive condition and should not be taken lightly. It is a potentially life-threatening condition that requires immediate medical attention. The risks of undiagnosed obstructive sleep apnea include heart attacks, strokes, impotence, irregular heartbeat, high blood pressure and heart disease. In addition, obstructive sleep apnea causes daytime sleepiness that can result in accidents, lost productivity and interpersonal relationship problems. The severity of the symptoms may be mild, moderate or severe.

Sleep apnea is diagnosed utilizing a sleep test, called polysomnography but treatment methodologies differ depending on the severity of the disorder. Mild Sleep Apnea is usually treated by some behavioral changes. Losing weight, sleeping on your side are often recommended. There are oral mouth devices (that help keep the airway open) that may help to reduce snoring in three different ways. Some devices (1) bring the jaw forward or (2) elevate the soft palate or (3) retain the tongue (from falling back in the airway and blocking breathing),

Moderate to severe Sleep Apnea is usually treated with a continuous positive airway pressure (C-PAP). C-PAP is a machine that blows air into your nose via a nose mask, keeping the airway open and unobstructed. For more severe apnea, there is a Bi-level (Bi-PAP) machine. The Bi-level machine is different in that it blows air at two different pressures. When a person inhales, the pressure is higher and in exhaling, the pressure is lower.

Some people have facial deformities that may cause the sleep apnea. It simply may be that their jaw is smaller than it should be or they could have a smaller opening at the back of the throat. Some people have enlarged tonsils, a large tongue or some other tissues partially blocking the airway. Fixing a deviated septum may help to open the nasal passages. Removing the tonsils and adenoids or polyps may help also. Children are much more likely to have their tonsils and adenoids removed. Surgical procedures, such as tracheostomy, ubulopalatopharyngoplasty (UPPP), laser assisted uvuloplasty (LAUP), somnoplasty, or mandibular myotomy, are often required to effectively treat sleep apnea.

### Depression

Recurrent mood disorders can have devastating long-term effects, and the cost of these illnesses in terms of human suffering, productivity and health care is enormous. It is now recognized that, for many patients, the long-term outcome is often much less favorable than previously thought, with incomplete interepisode recovery, and a progressive decline in overall functioning observed (Goldberg and Harrow, Psychopharmacol Bull, 1996;32(l): 47-54; Tohen et al., Am J Psychiatry. 2000 February; 157(2): 220-8). Indeed, according to the Global Burden of Disease Study, mood disorders are among the leading causes of disability worldwide, and are likely to represent an increasingly greater health, societal, and economic problem in the coming years (Murray and Lopez, Lancet. 1997; 349: 1436-42). Many antidepressants are currently available for the treatment of acute depression. Until a few decades ago, tricyclic antidepressants (TCAs) were the only drugs available for the treatment of depression. Monoamine oxidase inhibitors (MAOIs) were available and now, are seldom used. A number of new drugs followed in rapid succession, among them the selective serotonin reuptake inhibitors (SSRIs) and serotonin/norepinepherine reuptake inhibitors which are now widely used. Although options for pharmacologic treatment for depression have grown seemingly exponentially over the past several decades, the current armamentarium of antidepressants continues to have limitations of both efficacy and tolerability. Thus, there is a clear need to develop novel and improved therapeutics for major depression.

### Impulse Control Disorders

A large classification of mental disorders, broadly defined as Impulse Control Disorders (ICDs), are characterized by harmful behaviors performed in response to irresistible impulses. It has been suggested that ICDs may be related to obsessive-compulsive disorder or similarly, may be forms of obsessive-complusive disorders. It has also been hypothesized that ICDs may be related to mood disorder or may be forms of affective spectrum disorder, a hypothesized family of disorders sharing at least one common physiologic abnormality with major depression. In the Diagnostic and Statistical Manual of Mental Disorders (DSM-IV), the essential feature of an ICD is the failure to resist an impulse, drive, or temptation to perform an act that is harmful to the person or to others. For most ICDs, the individual feels an increasing sense of tension or arousal before committing the act, and then experiences pleasure, gratification, or release at the time of committing the act. After the act is performed, there may or may not be regret or guilt. ICDs are listed in a residual category, the ICDs Not Elsewhere Classified, which includes intermittent explosive disorder (IED), kleptomania, pathological gambling, pyromania, trichotillomania, and ICD not otherwise specified (NOS). Examples of ICDs NOS are compulsive buying or shopping, repetitive self-mutilation, nonparaphilic sexual addictions, severe nail biting, compulsive skin picking, personality disorders with impulsive features, attention deficit/hyperactivity disorder, eating disorders characterized by binge eating, and substance use disorders. Binge eating disorder (BED) is a subset of ICD which is characterized by discrete periods of binge eating during which large amounts of food are consumed in a discrete period of time and a sense of control over eating is absent. Persons with bulimia nervosa have been reported to have electroencephalographic abnormalities and to display reduced binge eating in response to the anti-epileptic drug phenytoin. Also, in controlled trials in patients with epilepsy, topiramate was associated with suppression of appetite and weight loss unrelated to binge eating.

### Alcohol Addiction

Alcohol addiction or alcohol dependence refers to a complex behavioral syndrome including abnormal importance of alcohol, the use of alcohol to an extreme and often harmful degree, continued use of alcohol despite negative consequences; psychological defenses of denial, rationalization, minimization and projection of blame and personality changes and life disruption as a consequence of use or activity. Alcohol addiction is characterized in a subject by the presence of one or more of the following symptoms. The subject has a tolerance for alcohol. The subject has withdrawal symptoms after stopping drinking alcohol. The subject takes alcohol in larger amounts than was intended. The subject lacks the ability to decrease the amount of alcohol consumed. The subject spends a great deal of time attempting to acquire alcohol. Lastly, the subject continues to use alcohol even though the subject should know that there are reoccurring physical or psychological problems being caused by the alcohol.

### Available Drug Therapies

Various combination therapies that include the sympathomimetic agent phentermine have been investigated and have met with mixed success. The phentermines were, up until around 1997, often prescribed along with fenfluramine (Pondimin®) or dexfenfluramine (Redux®), nicknamed fen, as a combination therapy known as fen-phen. Fenfluramine is a potent releaser of serotonin from serotonergic neurons which acts on a cerebral appetite center. When combined with phentermine, fenfluramine had the effect of enhancing and extending the anorexient action of phentermine. However in 1997, the Food and Drug Administration (“FDA”) asked manufacturers to withdraw Pondimin® and Redux® due to studies which strongly suggested that the drugs cause damage to the mitral valve of the heart and pulmonary hypertension.

More recently, it has been suggested that phentermine in combination with anti depressants is a potentially effective therapy for effecting weight loss, (U.S. Pat. No. 5,795,895). In particular, the anti-depressants suggested for use in this new combination therapy are members of a class of compounds known as selective serotonin reuptake inhibitors (SSRIs) which include fluoxetine (Prozac®), sertraline (Zoloft®), fluvoxamine maleate (Luvox®) and trazodone hydrochloride (Desyrel®). The combination therapy is also suggested to treat coexisting depression and/or obsessive-compulsive disorder.

Phentermine has also recently been tested in combination with bupropion (Wellbutrin®) for the treatment of obesity. Bupropion is an antidepressant that inhibits dopamine reuptake, as compared to serotonin uptake. It is also used to treat Attention Deficit Disorder (ADHD), bipolar depression, chronic fatigue syndrome, cocaine addiction, nicotine addiction, and lower back pain. While bupropion alone had a modest effect as a weight loss agent (when prescribed to patients following a 1200 calorie per day diet), patients receiving phentermine in combination with bupropion experienced no greater weight loss than those receiving bupropion alone. Moreover, bupropion use has been associated with drug-induced seizures causing it to be removed from the market by the FDA for at least five years before its re-introduction in 1989.

Zonisamide (ZONEGRAN™), a sulfonylurea antiepileptic drug, is used to control some kinds of seizures in the treatment of epilepsy. Zonisamide may produce these effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization. In vitro binding studies have demonstrated that zonisamide binds to the GABA/benzodiazepine receptor ionophore complex in an allosteric fashion which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity, but this pharmacologic effect is not thought to be a major contributing factor in the antiseizure activity of zonisamide. Side effects of zonisamide include but are not limited to: renal calculi, drowsiness, ataxia, loss of appetite, gastrointestinal symptoms, severe rash (i.e. Stevens Johnson Syndrome [SJS] and toxic epidermal necrolysis [TEN]), serious hematologic events, such as aplastic anemia or agranuclocytosis, oligohydrosis and hyperthermia in pediatric patients.

2,3,4,5-bis-O-(1-methylethylidene)-β-D-fructopyranose sulfamate, known as topiramate, is a member of a class of novel antiepileptic drugs and has been demonstrated in clinical trials of human epilepsy to be effective as adjunctive therapy or as monotherapy in treating simple and complex partial seizures and secondarily generalized seizures (E. Faught, et at. Karim et al., Epilepsia 1995, 36 (S4), 33; S. K. Sachdeo et al., Epilepsia 1995, 36 (S4), 33; T. A. Glauser, Epilepsia 1999, 40 (S5), S71-80; R. C. Sachdeo, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world. There has also been evidence that topiramate is effective in the treatment of diabetes (U.S. Pat. Nos. 7,109,174 and 6,362,220), neurological disorders (U.S. Pat. No. 6,908,902), depression (U.S. Pat. No. 6,627,653), psychosis (U.S. Pat. No. 6,620,819), headaches (U.S. Pat. No. 6,319,903) and hypertension (U.S. Pat. No. 6,201,010). However there have been adverse effects associated with the use of topiramate in humans.

All of the above mentioned drugs have shown some promise for having therapeutic effects in the above-mentioned pathologies. However, all of these drugs have adverse reactions that limit their ability to be used in humans at high doses. The instant invention is drawn to combinations of the drugs mentioned above and other drugs belonging to the drug families of the drugs mentioned above that maximize the therapeutic effect of these drugs and minimize their adverse effects.

### SUMMARY OF THE INVENTION

The present invention features a novel therapy for treating diabetes, hypertension, pulmonary hypertension, migraine, epilepsy, sleep apnea, depression, impulse control disorders or alcohol addiction which involves treating a subject with a sympathomimetic agent in combination with an anticonvulsant sulfamate compound or an anticonvulsant sulfonylurea compound. For example, the sympathomimetic agent is bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion). In one example, the sympathomimetic agent is the drug phentermine (nicknamed “phen”) or bupropion.

The phentermine may be, for example, administered at between about 2 mg and 60 mg per day. In one aspect, the phentermine is administered at between about 2 mg and 30 mg per day.

The bupropion may be, for example, administered at between about 50 mg and 400 mg per day. In one aspect, the bupropion is administered at between about 50 mg and 200 mg per day.

In one example, the anticonvulsant sulfamate compound is the drug topiramate. For example, topiramate is administered at between about 15 mg and 1500 mg per day. In one aspect, the topiramate is administered at between about 15 mg and 200 mg per day.

In another example, the anticonvulsant sulfonylurea compound is zonisamide. For example, zonisamide is administered at between about 20 mg and 400 mg per day. In one aspect, the zonisamide is administered at between about 20 mg and 200 mg per day.

The present invention also features a pharmaceutical composition that includes, e.g., bupropion in combination with an anticonvulsant sulfamate compound or an anticonvulsant sulfonylurea compound. The present invention also features a pharmaceutical composition that includes, e.g., phentermine in combination with an anticonvulsant sulfamate compound or an anticonvulsant sulfonylurea compound. For example, the pharmaceutical composition contains between about 2 mg and 60 mg phentermine. In one aspect, pharmaceutical composition contains between about 2 mg and 30 mg of phentermine. In another aspect, the pharmaceutical composition contains between about 50 mg and 400 mg bupropion. In another aspect, pharmaceutical composition contains between about 50 mg and 200 mg of bupropion.

For example, the pharmaceutical composition contains topiramate in combination with phentermine or bupropion. In one aspect, the pharmaceutical composition contains between about 15 mg and 1500 mg topiramate. In another aspect, pharmaceutical composition contains between about 15 mg and 200 mg of topiramate.

For example, the pharmaceutical composition contains zonisamide in combination with phentermine or bupropion. In one aspect, the pharmaceutical composition contains between about 20 mg and 200 mg zonisamide. In another aspect, pharmaceutical composition contains between about 20 mg and 200 mg of zonisamide.

The present invention also features a kit that includes bupropion or phentermine in combination with an anticonvulsant sulfamate compound or an anticonvulsant sulfonylurea compound in separate and discrete dosage forms, and instructions for its use. In one aspect, the anticonvulsant sulfamate compound is the drug topiramate. In another aspect, the anticonvulsant sulfonylurea compound is zonisamide.

### DETAILED DESCRIPTION OF THE INVENTION

It will be understood that particular embodiments described herein are shown by way of illustration and not as limitations of the invention. The principal features of this invention can be employed in various embodiments without departing from the scope of the invention. All parts and percentages are by weight unless otherwise specified. The scientific publications, patents or patent applications cited in the various sections of this document are herein incorporated-by-reference for all purposes.

The instant invention provides for the treatment of diabetes, hypertension, migraine, epilepsy, sleep apnea, depression, impulse control disorders or alcohol addiction and related disorders with a combination of one or more antiepileptic agents combined with one or more sympathomimetic agents. In one embodiment, the antiepileptic agent is either an anticonvulsant sulfamate compound or an anticonvulsant sulfonylurea compound. An example of an anticonvulsant sulfamate compound is topiramate. An example of an anticonvulsant sulfonylurea compound is zonisamnide. Sympathomimetic agents include bupropion, phenylethylamine, epinephrine, norepinephlrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion. In one embodiment, the sympathomimetic agent is phentermine or bupropion.

Evidence shows that topiramate has therapeutic effects on diabetes (U.S. Pat. Nos. 7,109,174 and 6,362,220) neurological disorders (U.S. Pat. No. 6,908,902), depression (U.S. Pat. No. 6,627,653), psychosis (U.S. Pat. No. 6,620,819), headaches (U.S. Pat. No. 6,319,903) and hypertension (U.S. Pat. No. 6,201,010). However there have been adverse effects associated with the use of topiramate in humans. To ameliorate these effects, it would be preferable to administer lower doses of topiramate.

Zonisamide has been shown to be effective in treatment of epileptic seizures and other mental disorders. Further, sulfonylurea drugs from this family have been shown to be effective in the treatment of diabetes. (U.S. Pat. No. 6,686,337). However, zonisamide has side effects associated with its administration to humans. It would be preferable to be able to dose zonisamide at a lower level and still have its therapeutic effects.

Phentermine has been shown to be effective in causing weight loss which is effective in decreasing the occurrence of diabetes and hypertension. Similarly bupropion has been shown to help increase weight loss alone or combined with phentermine. Further, bupropion has been shown effective in the treatment of depression, and other mental disorders. However both of these drugs have been shown to have adverse effects on patients at higher doses.

The combination of an anticonvulsant sulfamate compound like topiramate or an anticonvulsant sulfonylurea compound like zonisamide with one or more sympathomimetic agents such as phentermine and/or bupropion provide increased therapeutic effects, and reduced adverse effects, making these pharmaceutical combinations extremely effective therapeutics, especially in the treatment of diabetes, hypertension, migraine, epilepsy, sleep apnea, depression, impulse control disorders or alcohol addiction and related disorders.

### Definitions

For convenience, certain terms used in the specification, examples, and appended claims are collected here.

“Diabetes” is associated with continuous and pathologically elevated blood glucose concentration. There are different types of diabetes including type 1 and type 2 diabetes, gestational diabetes and pre-diabetes. Symptoms associated with diabetes include frequent urination, excessive thirst, extreme hunger, unusual weight loss, increased fatigue, irritability, blurry vision, obesity, overweight, high blood sugar, cardiovascular disease, blindness, retinopathy, neuropathy, hyperinsulinemia, lactic acidosis, hypertension, impaired glucose tolerance, and kidney damage.

“Hypertension” is a condition that occurs when the blood pressure inside the large arteries is too high. Pathologies and symptoms associated with hypertension are cardiovascular complications such as strokes, heart attacks, heart failure, heart rhythm irregularities, impaired vision, and kidney failure. Hypertensive disorders include pulmonary hypertension, primary pulmonary hypertension, and secondary pulmonary hypertension.

“Epilepsy” is defined as a brain disorder with recurrent, unprovoked seizures. Epilepsy includes seizures of focal onset and generalized seizures. The types of focal onset seizures are partial seizures of temporal lobe origin, frontal lobe origin or others. Focal epilepsies with genetic components include benign childhood epilepsy with centrotemporal spikes, childhood epilepsy with occipital paroxysms or primary reading epilepsy. The generalized genetic epilepsies include benign neonatal familial convulsions, benign neonatal convulsions, benign myoclonic epilepsy in infancy, childhood absence epilepsy, juvenile absence epilepsy, juvenile myoclonic epilepsy, and epilepsy with grand mal seizures on awakening. The cryptogenic childhood epilepsies with generalized seizures include infantile spasms without tuberous sclerosis, West syndrome, Lennox-Gastaut syndrome, epilepsy with myoclonic-astatic seizures, epilepsy with myoclonic absences, and symptomatic epilepsy such as infantile spasms associated with tuberous sclerosis, epilepsy with continuous spike and wave EEG during slow-wave sleep, and acquired epileptic aphasia (Landau-Kleffner syndrome).

The treatment of any convulsive disorders is also contemplated using the methods and compositions of the invention. These convulsive disorders include all forms of epilepsies, for example, temporal lobe epilepsy, focal epilepsies, including idiopathic epilepsies such as benign childhood epilepsy with centrotemporal spikes, childhood epilepsy with occipital paroxysms or primary reading epilepsy, symptomatic epilepsies with simple partial seizures, complex partial seizures, secondarily generalized seizures, generalized epilepsies and syndromes; generalized epilepsies including idiopathic epilepsies such as benign neonatal familial convulsions, benign neonatal convulsions, benign myoclonic epilepsy in infancy, childhood absence epilepsy, juvenile absence epilepsy, juvenile myoclonic epilepsy, and epilepsy with grand mal seizures on awakening; cryptogenic or symptomatic epilepsies including West syndrome, Lennox-Gastaut syndrome, epilepsy with myoclonic-astatic seizures, epilepsy with myoclonic absences, and symptomatic epilepsy such as early myoclonic encephalopathy or specific syndromes; epilepsies with undetermined origin including neonatal seizures, severe myoclonic epilepsy in infancy, epilepsy with continuous spike and wave EEG during slow-wave sleep, and acquired epileptic aphasia (Landau-Kleffner syndrome). It also includes seizures caused by metabolic derangements, acute brain injury, acute head trauma, drug withdrawal, alcohol withdrawal, and toxins.

Symptoms associated with, or arising from epilepsy, include convulsions, grand mal seizures, absence seizures, petit mal seizures, focal seizures, temporal lobe seizures, psychomotor seizures, muscle spasms, loss of consciousness, strange sensations, strange emotions and strange behavior.

“Sleep apnea” is a condition which can be defined as obstructive sleep apnea, central sleep apnea or mixed sleep apnea. Obstructive sleep apnea is the most common form which occurs when throat muscles relax. Central sleep apnea occurs when your brain doesn't send proper signals to the muscles that control breathing. Mixed sleep apnea is a combination of both obstructive and central sleep apneas. Sleep apnea is characterized by repetitive episodes of upper airway obstruction that occur during sleep, usually associated with a reduction in blood oxygen saturation. The airway becomes obstructed at several possible sites. The upper airway can be obstructed by excess tissue in the airway, large tonsils, a large tongue and usually includes the airway muscles relaxing and collapsing when asleep. Another site of obstruction can be the nasal passages. Sometimes the structure of the jaw and airway can be a factor in sleep apnea.

Symptoms associated with, or arising from sleep apnea, include insomnia, hypersomnia, loud snoring; episodes of breathing cessation during sleep, abrupt awakenings accompanied by shortness of breath; dry mouth or sore throat, and/or morning headache.

“Migraines” are generally headaches that are typically associated with various psychological (e.g., irritability, depression, fatigue, drowsiness, and restlessness), neurological (e.g., photophobia, and phonophobia), and gastrointestinal symptoms. The headache starts with mild pain, which increases in intensity over a short period of time. There are two major types of migraines. The common migraine affects 80-85% of migraine sufferers and classical migraine with aura affects 15% of migraine sufferers. Symptoms associated with migraines include headaches, psychological symptomatology such as irritability, depression, fatigue, drowsiness, restlessness; neurological symptoms such as photophobia, phonophobia or gastrointestinal symptoms such as change in bowel habit, change of food intake or urinary symptoms such as urinary frequency, auras which are neurological deficits and can be a variety of deficits for the migraine population but in the individual is usually stereotyped. These deficits may be visual scotoma or visual designs, hemiplegia, migrating paraesthesia, dysarthria, dysphasia, or deja-vu. The headache is usually accompanied by light or sound sensitivity, photophobia or phonophobia, irritability and impaired concentration.

“Depression” is manifested by a combination of symptoms that interfere with the ability to work, study, sleep, eat, and enjoy once pleasurable activities. Depression includes major depression, especially refractory depression, bipolar depression, and the degeneration associated with depression. Symptoms of depression include persistent sad, anxious, or “empty” mood, feelings of hopelessness, pessimism, feelings of guilt, worthlessness, helplessness, loss of interest or pleasure in hobbies and activities that were once enjoyed, including sex, decreased energy, fatigue, being “slowed down”, difficulty concentrating, remembering, making decisions, insomnia, early-morning awakening, or oversleeping, appetite and/or weight loss or overeating and weight gain, thoughts of death or suicide; suicide attempts, restlessness, irritability, persistent physical symptoms that do not respond to treatment, such as headaches, digestive disorders, and chronic pain.

Other psychiatric disorders may also be treated using the compositions and methods of the invention. These disorders include panic syndrome, general anxiety disorder, phobic syndromes of all types, mania, manic depressive illness, hypomania, unipolar depression, stress disorders, PTSD, somatoform disorders, personality disorders, psychosis, and schizophrenia.

“Impulse Control Disorders” are characterized by harmful behaviors performed in response to irresistible impulses. The essential feature of an impulse control disorder is the failure to resist an impulse, drive, or temptation to perform an act that is harmful to the person or to others. Symptoms include an increasing sense of tension or arousal before committing an act, and then experiences pleasure, gratification, or release at the time of committing the act. After the act is performed, there may or may not be regret or guilt. Numerous disorders can be characterized as impulse control disorders including intermittent explosive disorder, kleptomania, pathological gambling, pyromania, trichotillomania, compulsive buying or shopping, repetitive self-mutilation, nonparaphilic sexual addictions, severe nail biting, compulsive skin picking, personality disorders with impulsive features, attention deficit/hyperactivity disorder, eating disorders characterized by binge eating, and substance use disorders.

“Alcohol addiction” is characterized in a subject by the presence of one or more of the following symptoms. The subject has a tolerance for alcohol. The subject has withdrawal symptoms after stopping drinking alcohol. The subject takes alcohol in larger amounts than was intended. The subject lacks the ability to decrease the amount of alcohol consumed. The subject spends a great deal of time attempting to acquire alcohol. Lastly, the subject continues to use alcohol even though the subject should know that there are reoccurring physical or psychological problems being caused by the alcohol.

Symptoms associated with alcohol addiction include death from alcohol toxemia, cirrhosis of the liver, pancreatitis, heart disease, polyneuropathy, alcoholic dementia, increased incidence of many types of cancer: breast cancer, head and neck cancer, esophageal cancer and colorectal cancer, nutritional deficiency involving deficiencies in folic acid, thiamine (vitamin B1), sexual dysfunction, osteoporosis and osteonecrosis.

As used herein, the term “Agent” includes a protein, polypeptide, peptide, nucleic acid (including DNA or RNA), antibody, small molecule, compound, antibiotic, or-drug, and any combinations thereof.

As used herein, the term “Antiepileptic Agents” includes molecules useful for the treatment of epilepsy. Examples include GABA-T inhibitors like γ-vinyl GABA (vigabatrin). Anti-epileptic agents include carbamazepine, clonazepam, ethosuximide, gabapentin, lamotrigine, levetiracetam, phenobarbital, phenytoin, primidone, tiagabine, topiramate, valproate, felbamate, oxazinane-dione, metharbital, ethotoin and mesantoin. Antiepileptirc gents also include vigabatrin, gabapentin, and oxcarbazepine. Antiepileptic agents also include anticonvulsant sulfamate compounds and anticonvulsant sulfonylurea compounds as further defined below.

“Treating”, includes any effect, e.g., lessening, reducing, modulating, or eliminating, that results in the improvement of the condition, disease, disorder, etc. In one embodiment, effective treatment is defined here as no seizures and no side effects.

Preferably, the term “Subject” refers to a mammal. More preferably, the term subject refers to a primate. More preferably, the term subject refers to a human.

“Pharmaceutically or Pharmacologically Acceptable” include molecular entities and compositions that do not produce an adverse, allergic or other untoward reaction when administered to an animal, or a human, as appropriate.

“Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

“Pharmaceutically Acceptable Salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The phrase “therapeutically effective amount” as used herein refers to the amount of an agent, compound, drug, composition, or combination of the invention which is effective for producing some desired therapeutic effect upon administration to a subject or patient (e.g., a human subject or patient).

The phrase administering to a subject or administering to a patient refers to the process of introducing an agent, compound, drug, composition or combination of the invention into the subject or patient's body via an art-recognized means of introduction (e.g, orally, transdermally, via injection, etc.).

The term sympathomimetic agent or sympathomimetic drug is a term of art and refers to agents or compounds which mimic or alter stimulation of the sympathetic nervous system (e.g., stimulates the peripheral nervous system) of an organism (e.g., mimic the stimulation naturally effected by physical activity, psychological stress, generalized allergic reaction and other situations in which the organism is provoked).

Sympathomimetic agents for use in the present invention and their general clinical uses or effects are set forth in Table I.

TABLE I
Sympathomimetic Agents and Clinical Uses Thereof

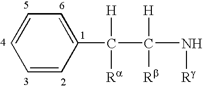

| General structure: | | Main Clinical Uses | | | | | |
|---|---|---|---|---|---|---|---|
| | Ring | | | | α Receptor | β Receptor | |
| Agent name | substituent(s) | R^{α} | R^{β} | R^{γ} | A N P V | B C | CNS, 0 |
| Bupropion | 3-Cl | ═O | CH₃ | C(CH₃)₃ | | | |
| Phenylethylamine | | H | H | H | | | |
| Epinephrine | 3-OH, 4-OH | OH | H | CH₃ | A, P, V | B, C | |
| Norepinephrine | 3-OH, 4-OH | OH | H | H | P | | |
| Epinine | 3-OH, 4-OH | H | H | CH₃ | | | |
| Dopamine | 3-OH, 4-OH | H | H | H | P | | |
| Dobutamine | 3-OH, 4-OH | H | H | 1* | | C | |
| Nordefrin | 3-OH, 4-OH | OH | CH₃ | H | V | | |
| Ethylnorepinephrine | 3-OH, 4-OH | OH | CH₂CH₃ | H | | B | |
| Isoproterenol | 3-OH, 4-OH | OH | H | CH(CH₃)₂ | | B, C | |
| Protokylol | 3-OH, 4-OH | OH | H | 2* | | B | |
| Isoetharine | 3-OH, 4-OH | OH | CH₂CH₃ | CH(CH₃)₂ | | B | |
| Metaproterenol | 3-OH, 5-OH | OH | H | CH(CH₃)₂ | | B | |
| Terbutaline | 3-OH, 5-OH | OH | H | C(CH₃)₃ | | B | |
| Metaraminol | 3-OH | OH | CH₃ | H | P | | |
| Phenylephrine | 3-OH | OH | H | CH₃ | N, P | | |
| Tyramine | 4-OH | H | H | H | | | |
| Hydroxyamphetamine | 4-OH | H | CH₃ | H | N, P | C | |
| Methoxyphenamine | 2-OCH₃ | H | CH₃ | CH₃ | | B | |
| Methoxamine | 2-OCH₃,5- | OH | CH₃ | H | P | | |
| | OCH₃ | | | | | | |
| Albuterol | 3-CH₂OH, 4- | OH | H | C(CH₃)₃ | | B | |
| | OH | | | | | | |
| Amphetamine | | H | CH₃ | H | | | CNS, 0 |
| Methamphetamine | | H | CH₃ | CH₃ | P | | CNS, 0 |
| Benzphetamine | | H | CH₃ | —NHR^{γ} | | | 0 |
| | | | | is replaced | | | |
| | | | | with 3* | | | |
| Ephedrine | | OH | CH₃ | CH₃ | N, P | B, C | |
| Phenylpropanolamine | | OH | CH₃ | H | N | | |
| Mephentermine | | H | —CHR^{β}— is | CH₃ | N, P | | |
| | | | replaced | | | | |
| | | | with 4* | | | | |
| Phentermine | | H | —CHR^{β}— is | H | | | 0 |
| | | | replaced | | | | |
| | | | with 4* | | | | |
| Chlorphentermine | 4-Cl | H | —CHR^{β}— is | H | | | 0 |
| | | | replaced | | | | |
| | | | with 4* | | | | |
| Fenfluramine | 3-CF₃ | H | CH₃ | C₂H₅ | | | 0 |
| Propylhexedrine | 5*: phenyl ring | H | CH₃ | CH₃ | N | | |
| | is replaced with | | | | | | |
| | cyclohexyl | | | | | | |

| | | |
|---|---|---|
| Diethylpropion | 6*: The substituent at the 1- | 0 |
| | position is replaced with 6, | |
| | below. | |
| Phenmetrazine | 7*: The substituent at the 1- | 0 |
| | position is replaced with 7, | |
| | below. | |
| Phendimetrazine | 8*: The substituent at the 1- | 0 |
| | position is replaced with 8, | |
| | below. | |

| | | |
|---|---|---|
| 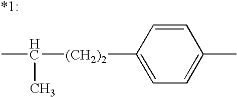 | 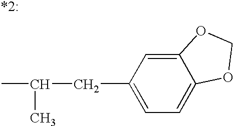 | 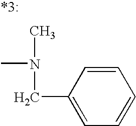 |

| | | | | |
|---|---|---|---|---|
| 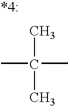 | 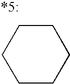 | 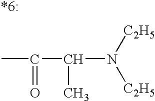 | 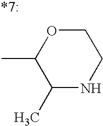 | 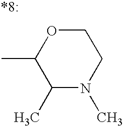 |

| | | |
|---|---|---|
| α Activity | β Activity | |
| A = Allergic reactions (includes β action) | B = Bronchodilator | CNS = Central nervous system |
| N = Nasal decongestion | C = Cardiac | 0 = Anorectic |
| P = Pressor (may include β action) | | |
| V = Other local vasoconstriction | | |
| (e.g in local anesthesia) | | |

*Numbers bearing an asterisk refer to the substituents numbered in the bottom rows of the table; substituent 5
replaces the phenyl rings, and 6, 7 and 8 are attached directly to the phenyl ring, replacing the ethylamine side chain.
^{†}The α and β in the prototype formula refer to positions of the C atoms in the ethylamine side chain.

In certain embodiments, the sympathomimetic agent is phentermine or a phentermine-like compound As defined herein, a phentermine-like compound is a compound structurally related to phentermine (e.g., an analog or derivative) which maintains an anorectic activity similar to that of phentermine. One phentermine-like compound is chlorphentermine. In yet another embodiment, the sympathomimetic agent is amphetamine or an amphetamine-like compound. As used herein, an amphetamine-like compound is a compound structurally related to amphetamine (e.g., an analog or derivative) which maintains an anorectic effect of amphetamine. In yet another embodiment, the sympathomimetic agent is phenmetrazine or a phenmetrazine-like compound. As defined herein, a “phenmetrazine-like compound” is a compound structurally related to phenmetrazine (e.g., an analog or derivative) which maintains an anorectic effect of phenmetrazine. One phenmetrazine-like compound is phendimetrazine. Analogs and/or derivatives of the compounds of the present invention can be tested for their ability to suppress appetite (e.g., suppress food intake) in a subject (e.g., a mammalian subject).

In other embodiments, the sympathomimetic agent is bupropion or a bupropion-like compound. As defined herein, a bupropion-like compound is a compound structurally related to bupropion (e.g., an analog or derivative) which maintains an anti-depressive activity similar to that of bupropion

In an exemplary embodiment, the sympathomimetic agent is selected from bupropion, amphetamine, methamphetamine, benzphetamine, phenylpropanolamine, phentermine, ehlorphentermine, diethylpropion, phenmetrazine, and phendimetrazine (as set forth in Table I). In one embodiment, the sympathomimetic agent is phentermine. It is also within the scope of the present invention to utilize other sympathomimetic agents including pseudo ephedrine (a stereoisomer of ephedrine, SUDAFED®), methylphenidate (RITALIN®), tuaminoheptane, other CNS stimulants including, for example, caffeine and bupropion.

The terms “anticonvulsant sulfamate compound (s),” “anticonvulsant sulfamate agent(s)” “nticonvulsant sulfamate derivative(s)” or “anticonvulsant sulfamate drug(s)” are terms of art and refer to a class of sulfamate-derived compounds that possess anticonvulsant activity and have an art-recognized use in the treatment of epilepsy. In particular, the anticonvulsant sulfamate compounds are monosaccharide derivatives with sulfamate functionality. The anticonvulsant sulfamate compounds for use in the present invention have one or more of the following modes of activity: modulation of voltage-dependent sodium conductance; potentiation of gamma-aminobutyric acid-evoked currents; inhibition of the kainate/alpha-amino-3-hydroxy-5-methylisoxazole-4-propionic acid (AMPA) subtype of the glutamate receptor; and/or inhibition of carbonic anhydrase (e.g., a mechanism by which the anticonvulsant derivative of the present invention may decrease the sensation of taste). The anticonvulsant sulfamate compounds for use in the present invention are described further in U.S. Pat. Nos. 4,513,006, 5,384,327, 5,498,629, 5,753,693 and 5,753,694, as are methods of synthesizing such anticonvulsant sulfamate compounds. The aforementioned patents are incorporated by reference herein in their entireties.

In certain embodiments, the anticonvulsant sulfamate compound is a compound having the following formula (I):

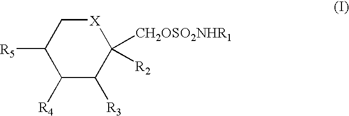

wherein:

X is CH₂ or O;

R₁ is H or alkyl; and

R₂, R₃, R₄ and R₅ are independently H or lower alkyl, with the proviso that when X is O, then R₂ and R₃ and/or R₄ and R₅ together may be a methylenedioxy group of the following formula (II):

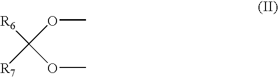

in which R₆ and R₇ are the same or different and are H or lower alkyl, or are joined to form a cyclopentyl or cyclohexyl ring.

R₁ in particular is hydrogen or alkyl of about 1 to 4 carbons, such as methyl, ethyl, or isopropyl. Alkyl includes both straight and branched chain alkyl. Alkyl groups R₂, R₃, R₄, R₅, R₆ and R₇ are about 1 to 3 carbons and include methyl, ethyl, isopropyl and n-propyl.

A particular group of compounds of the formula (I) are those wherein X is oxygen and both R₂ and R₃, and R₄ and R₅ together are methylenedioxy groups of the formula (II), wherein R₆ and R₇ are both hydrogen, both alkyl, or combine to form a spiro cyclopentyl or cyclohexyl ring, in particular, where R₆ and R₇ are both alkyl such as methyl. A second group of compounds are those wherein X is CH₂ and R₄ and R₅ are joined to form a benzene ring. A third group of compounds of the formula (I) are those wherein both R₂ and R₃ are hydrogen.

In one embodiment, the anticonvulsant sulfamate compound is topiramate (Topamax®). Topiramate, also referred to in the art as 2,3,4,5-bis-O-(1-methylethylidene)-β-D-fructopyranose sulfamate, has been demonstrated in clinical trials of human epilepsy to be effective as an adjunctive therapy or as monotherapy in treating simple and complex partial seizures and secondarily generalized seizures (E Faught et at. (1995) Epitepsia 36(suppl 4):33; S. Sachdeo et at, (1995) Epilepsia 36(suppl 4):33) and is currently marketed for the treatment of simple and complex partial seizure epilepsy with or without secondary generalized seizures.

In another embodiment, the sulfamate compound is selected from 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate and (S)-4,5-O-[N-(1,1-dimethylethoxycarbonyl)imidosulfinyl]-2,3-O-(1-methylethy lidene)-beta-D-fructopyranose sulfamate.

The terms “anticonvulsant sulfonylurea compound(s),” “anticonvulsant sulfonylurea agent(s),” “anticonvulsant sulfonylurea derivative(s),” or “anticonvulsant sulfonylurea drug(s)” are terms of art and refer to a class of sulfonylurea-derived compounds that possess anticonvulsant activity and have an art-recognized use in the treatment of epilepsy. In one embodiment the anticonvulsant sulfonylurea compound is zonisamide. Zonisamide (ZONEGRAN™), a sulfonylurea antiepileptic drug, is used to control some kinds of seizures in the treatment of epilepsy. Zonisamide may produce these effects through action at sodium and calcium channels. Other sulfonylurea antiepileptic drugs include chlorpropamide, tolazamide, tolbutamide, glyburide, glipizide and glimepiride.

### Dosages, Administration and Pharmaceutical Compositions:

The choice of appropriate dosages for the drugs used in combination therapy according to the present invention can be determined and optimized by the skilled artisan, e.g., by observation of the patient, including the patient's overall health, the response to the combination therapy, and the like. Optimization, for example, may be necessary if it is determined that a patient is not exhibiting the desired therapeutic effect or conversely, if the patient is experiencing undesirable or adverse side effects that are too many in number or are of a troublesome severity.

Preferably, a sympathomimetic drug (e.g., a drug set forth in Table I) is prescribed at a dosage routinely used by the skilled artisan (e.g., physician) to promote the desired therapeutic effect of the drug, when the drug is used as a monotherapy. For example, an anticonvulsant sulfamate compound (e.g., a compound having formula I) and/or an anticonvulsant sulfonylurea compound is prescribed at a lower dosage than routinely used by the skilled artisan (e.g., physician) to promote the desired therapeutic effect of the drug, when the drug is used as a monotherapy (e.g., in the treatment of epilepsy).

In one embodiment, each component of the combination (e.g., a sympathomimetic drug and anticonvulsant sulfamate compound or anticonvulsant sulfonylurea compound) is prescribed at a dose that is below the typically described dose for each component as a monotherapy. The components may be prescribed separately or as a combination dosage.

In one embodiment, each component of the combination (e.g., a sympathomimetic drug and anticonvulsant sulfamate compound or anticonvulsant sulfonylurea compound) is prescribed at a dose that is above the typically described dose for each component as a monotherapy. The components may be prescribed separately or as a combination dosage.

In another embodiment, the prescribed dosage of the sympathomimetic drug is above the typically described dose for monotherapy, and the anticonvulsant sulfamate compound or anticonvulsant sulfonylurea compound is prescribed at a dosage that is at or below the typically described dose for monotherapy. In another embodiment, the prescribed dosage of the sympathomimetic drug is at or below the typically described dose for monotherapy, and the anticonvulsant sulfamate compound or anticonvulsant sulfonylurea compound is prescribed at a dosage that is above the typically described dose for monotherapy.

It is especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” of an active compound calculated to produce the desired therapeutic effect in association with the required pharmaceutical carrier. The specifications of the novel dosage unit forms of the invention are dependent on the unique characteristics of the composition containing the anticonvulsant or sympathomimetic agent and the particular therapeutic effect to be achieved. Dosages can further be determined by reference to the usual dose and manner of administration of the ingredients. It is also within the scope of the present invention to formulate a single physically discrete dosage form having each of the active ingredients of the combination treatment (e.g., a single dosage form having an anticonvulsant agent and a sympathomimetic agent).

The method of administration of compositions or combinations of the invention will depend, in particular, on the type of sympathomimetic agent used and the chosen anticonvulsant sulfamate compound. The sympathomimetic agent and the anticonvulsant sulfamate compound or anticonvulsant sulfonylurea compound may be administered together in the same composition or simultaneously or sequentially in two separate compositions. Also, one or more sympathomimetic agents or one or more anticonvulsant sulfamate compounds or one or more anticonvulsant sulfonylurea compounds may be administered to a subject or patient either in the form of a therapeutic composition or in combination, e.g., in the form of one or more separate compositions administered simultaneously or sequentially, The schedule of administration will be dependent on the type of sympathomimetic agent(s) and anticonvulsant sulfamate compound (s) and anticonvulsant sulfonylurea compound (s) chosen. For example, a sympathomimetic agent can have a stimulant effect and the degree of such stimulant effect may vary depending on the sympathomimetic agent chosen. Accordingly, a sympathomimetic agent having a significant stimulant effect might be administered earlier in the day than administration of a sympathomimetic agent having a lesser stimulant effect. Likewise, an anticonvulsant sulfamate compound or an anticonvulsant sulfonylurea compound can have a sedative effect and the degree of such sedative effect may vary depending on the anticonvulsant sulfamate compound chosen. Accordingly, an anticonvulsant sulfamate compound or a anticonvulsant sulfonylurea compound having a significant sedative effect might be administered later in the day than administration of an anticonvulsant sulfamate compound having a lesser sedative effect. Moreover, sympathomimetic agents and/or anticonvulsant agents having lesser stimulant or sedative effects, respectively, may be administered simultaneously.

Sympathomimetic agents and/or anticonvulsant sulfamate compounds and/or anticonvulsant sulfonylurea compound scan also be administered along with a pharmaceutically acceptable carrier. As used herein “pharmaceutically acceptable carrier” includes any solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in compositions of the invention is contemplated.

A sympathomimetic agent alone, or in combination with an anticonvulsant sulfamate compound or an anticonvulsant sulfonylurea compound in the form of a-composition, is preferably administered orally. When the composition(s) are orally administered, an inert diluent or an assimilable edible carrier may be included. The composition and other ingredients may also be enclosed in a hard or soft shell gelatin capsule, compressed into tablets, or incorporated directly into the individual's diet. For oral therapeutic administration, the composition may be incorporated with excipients and used in the form of ingestible tablets, buccal tablets, troches, capsules, elixirs, suspensions, syrups, wafers, and the like. The percentage of the compositions and preparations may, of course, be varied. The amount of active compound in such therapeutically useful compositions is such that a suitable dosage will be obtained. In a particular embodiment, the present invention includes pharmaceutical composition comprising a therapeutically effective amount of a sympathomimetic agent and an anticonvulsant sulfamate compound or an anticonvulsant sulfonylurea compound. In one embodiment, the present invention includes a therapeutically-effective amount of a sympathomimetic agent and an anticonvulsant sulfamate compound or an anticonvulsant sulfonylurea compound packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

The tablets, troches, pills, capsules and the like may also contain a binder, an excipient, a lubricant, or a sweetening agent. Various other materials may be present as coatings or to otherwise modify the physical form of the dosage unit. For instance, tablets, pills, or capsules may be coated with shellac, sugar or both. Of course, any material used in preparing any dosage unit form should be pharmaceutically pure and substantially non-toxic in the amounts employed.

A sympathomimetic agent, alone or in combination with an anticonvulsant sulfamate compound or an anticonvulsant sulfonylurea compound, can also be administered in a convenient manner such as by injection (subcutaneous, intravenous, etc.), inhalation, transdermal application, or rectal administration. Depending on the route of administration, the composition containing the sympathomimetic agent and/or anticonvulsant sulfamate compound and/or anticonvulsant sulfonylurea compound may be coated with a material to protect the compound from the action of acids and other natural conditions which may inactivate the compounds or compositions.

To administer the compositions, for example, transdermally or by injection, it may be necessary to coat the composition with, or co-administer the composition with, a material to prevent its inactivation. For example, the composition may be administered to an individual in an appropriate diluent or in an appropriate carrier such as liposomes. Pharmaceutically acceptable diluents include saline and aqueous buffer solutions. Liposomes include water-in-oil-in-water CGF emulsions as well as conventional liposomes (Strejan el al. (1984) J. Neuroimmunol. 7:27). To administer the compositions containing the sympathomimetic agents and/or anticonvulsant sulfamate compounds and/or anticonvulsant sulfonylurea compounds parenterally or intraperitoneally, dispersions can be prepared in glycerol, liquid polyethylene glycols, and mixtures thereof and in oils. Under ordinary conditions of storage and use, these preparations may contain a preservative to prevent the growth of microorganisms.

Compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils, The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents. In many cases, it will be preferable to include isotonic agents, for example, sugars, polyalcohols such as mannitol and sorbitol, or sodium chloride in the composition. Prolonged absorption of the injectable compositions can be brought about by including in the composition an agent which delays absorption, for example, aluminum monostearate and gelatin.

One aspect of the present invention features prescribing phentermine in combination with topiramate or zonisamide to treat diabetes, hypertension, migraine, epilepsy, depression or alcohol addiction. A typical dose for phentermine is between about 2-60 mg daily, including but not limited to doses of 2, 3, 4, 5, 6, 7, 8, 10, 15, 20, 25, 30, 35, 40, 45, 50 and 55 mg daily. One dose for phentermine is about 15 mg daily. In an embodiment, the phentermine is of an immediate release form.

Typically, the phentermine is taken by the patent in the morning, for example, it is taken before breakfast. The phentermine is best taken in the morning because the drug is a stimulant. When phentermine is prescribed (e.g., as part of the combination therapy described herein), physicians should be aware and may want to advise patients that the drug can be mildly habit forming. Phentermine can also cause increased nervousness, increased energy, irritability and, rarely, insomnia. Stopping phentermine may also cause tiredness lasting for up to 1-2 weeks. Phentermine can also raise blood pressure (e.g., during the early phases of treatment).

Another aspect of the present invention features prescribing bupropion in combination with topiramate to treat diabetes, hypertension, migraine, epilepsy, depression or alcohol addiction. A typical dose for bupropion is between about 10-500 mg daily, including but not limited to doses of 15, 50, 100, 150, 200, 250, 300, 350, 400, 450 and 475 mg daily. For example, a typical dose for bupropion is between about 50-400 mg daily. One dose for bupropion is about 200 mg daily.

A typical dose for topiramate is between about 50-1500 mg daily. As discussed previously, prescription of topiramate at dosages of≧400 mg daily results is promotion of undesirable side effects (e.g., sedation, mental clouding). Accordingly, in a one embodiment, topiramate is prescribed at a dose of about 50-400 mg daily. In one embodiment, topiramate is prescribed at a dose of about 15-200 mg daily In another embodiment, the dosage of topiramate is increased gradually at the outset of the therapy in order to reduce the chance of undesirable side effects associated with higher doses of the drug. In an embodiment, the topiramate is administered at a dose of 25 mg daily for about the first 5-7 days (e.g, 6 days) of treatment, at a dose of about 50 mg daily for the next 5-7 days (e g., 6 days), at a dose of 100 mg daily for about the next 6-8 days (e.g., 7 days) and about 150 mg daily for the next 20-26 days. From this point forward, the topiramate can be administered at a dose of 150-250 mg daily, including but not limited to doses of 175, 200, and 225 mg daily. One dose for continued therapy is about 200 mg of topiramate daily. In another embodiment, the topiramate is of an immediate release form. In yet another embodiment, the topiramate is of a sustained release form.

A typical dose of zonisamide is between 20 and 500 mg daily, for example between 20 and 400 mg daily. Less than 400 mg/day is preferred. Doses of zonisamide include, but are not limited doses of 20, 30, 40, 50, 100, 150, 200, 250, 300, 350, 400, 450 and 475 mg daily.

In one embodiment, topiramate is taken later in the day than the phentermine. For example, the patient takes the topiramate just before supper or later in the evening. Topiramate is best given later in the day because the drug can be sedating. In other embodiments, the topiramate is given BID (e.g., twice daily), TID (three times daily) or QID (four time daily). When prescribing topiramate, physicians should be aware and may want to advise patients that the drug can cause tiredness, fatigue, dizziness, difficulty with speech or finding words, difficulty concentrating, difficulty with balance, and/or numbness or tingling in the hands or feet. Less common side effects are nausea, coordination problems, abdominal pain, slowed thinking nervousness, depression, breast pain, painful periods, double or blurred vision, palpitations, low white blood count and kidney stones. A physician should also advise patients that the drug may not be taken if the patient is also taking Diamox (acetazolamide). No female patient should become pregnant while taking this drug as it may cause birth defects. If a female patient misses a period she should immediately discontinue taking the medication and inform the physician. Female patients should not be treated according to the methods of the present invention if breast feeding a child. Patients should not drink alcohol or take sedating medications while taking topiramate since excess sedation can occur. Patients should also refrain from performing dangerous tasks (e.g,. operating heavy machinery or driving) until they are comfortable with the side effects of the full dose (e.g, 200-400 mg daily). Patients should be advised not to increase the dosage beyond what is prescribed. Topiramate is not habit forming.

Yet another embodiment of the present invention features pharmaceutical compositions (e.g, for oral administration) comprising phentermine and topiramate in a single pharmaceutical formulation. Such compositions may be preferred, for example, to increase patient compliance (e.g., by reducing the number of administrations necessary to achieve the desired pharmacologic effect).

Yet another embodiment of the present invention features pharmaceutical compositions (e.g., for oral administration) comprising bupropion and topiramate in a single pharmaceutical formulation. Such compositions may be preferred, for example, to increase patient compliance (e.g., by reducing the number of administrations necessary to achieve the desired pharmacologic effect).

Yet another embodiment of the present invention features pharmaceutical compositions (e.g., for oral administration) comprising bupropion and zonisamide in a single pharmaceutical formulation. Such compositions may be preferred, for example, to increase patient compliance (e.g., by reducing the number of administrations necessary to achieve the desired pharmacologic effect).

Yet another embodiment of the present invention features pharmaceutical compositions (e.g., for oral administration) comprising phentermine and zonisamide in a single pharmaceutical formulation. Such compositions may be preferred, for example, to increase patient compliance (e.g., by reducing the number of administrations necessary to achieve the desired pharmacologic effect).

In an embodiment, the pharmaceutical composition includes phentermine in an immediate release form and further includes topiramate in a controlled release formulation. As defined herein, an “immediate release formulation” is one which has been formulated to allow, for example, the phentermine, to act as quickly as possible, Immediate release formulations include, but are not limited to, readily dissolvable formulations. As defined herein, a “controlled release formulation” includes a pharmaceutical formulation that has been adapted such that drug release rates and drug release profiles can be matched to physiological and chronotherapeutic requirements or, alternatively, has been formulated to effect release of a drug at a programmed rate. Controlled release formulations include, but are not limited to, granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed therethrough), granules within a matrix, polymeric mixtures, granular masses, and the like.

In one embodiment, a controlled release formulation is a delayed release form. As defined herein, a “delayed release form” is formulated in such a way as to delay, for example, topiramate's action for an extended period of time. A delayed release form can be formulated in such a way as to delay the release of an effective dose of topiramate for 4, 8, 12, 16 or 24 hours following the release of phentermine. In yet another embodiment, a controlled release formulation is a sustained release form. As defined herein, a “sustained release form” is formulated in such a way as to sustain, for example, the topiramate's action over an extended period of time. A sustained release form can be formulated in such a way as to provide an effective dose of topiramate (e.g., provide a physiologically effective blood level) over a 4, 8, 12, 16 or 24 hour period.

Compositions include a tablet core that includes topiramate, said core being in association with a layer of phentermine. For example, the core has a delayed or sustained dissolution rate. In an exemplary embodiment, a tablet can comprise a first layer containing, for example, phentermine (e.g., in an immediate release formulation) and a core containing, for example, topiramate in a delayed release or sustained release formulation. Other exemplary embodiments can include, for example, a barrier between the first layer and core, said layer serving the purpose of limiting drug release from the surface of the core. Preferred barriers prevent dissolution of the core when the pharmaceutical formulation is first exposed to gastric fluid. For example, a barrier can comprise a disintegrant, a dissolution-retarding coating (e.g., a polymeric material, for example, an enteric polymer), or a hydrophobic coating or film, and can be selectively soluble in either the stomach or intestinal fluids. Such barriers permit the topiramate to leach out slowly and can cover substantially the whole surface of the core.

The above-described pharmaceutical compositions are designed to release the two effective agents of the combination therapy of the present invention sequentially, i.e., releasing topiramate after releasing phentermine, both agents being contained in the same pharmaceutical composition. Exemplary amounts of phentermine and topiramate are as described above with certain compositions comprising from about 2 mg to about 60 mg phentermine and from about 15 mg to 1500 mg topiramate. Certain compositions include at least 15 mg phentermine and at least about 50 mg, 100 mg or 200 mg topiramate.

Pharmaceutical compositions so formulated may contain additional additives, suspending agents, diluents, binders or adjuvants, disintegrants, lubricants, glidants, stabilizers, coloring agents, flavors, etc. These are conventional materials which may be incorporated in conventional amounts.

### Additivity/Synergy

Compositions of one or more sympathomimetic agents in combination with one or more anticonvulsant sulfamate compounds and/or one or more anticonvulsant sulfonylurea compounds are synergistically effective and are effective in treating a convulsive disorder such as diabetes, hypertension, migraine, epilepsy or depression.

Synergy is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is 75%, the effect of A and B is synergistic.

Additivity is defined as the interaction of two or more agents so that their combined effect is more than each individually and as much as the sum of their individual effects. For example, if the effect of drug A alone in treating a disease is 25%, and the effect of drug B alone in treating a disease is 25%, but when the two drugs are combined the effect in treating the disease is greater than 25% and as high as 50%, the effect of A and B is additive.

An improvement in the drug therapeutic regimen can be described as the interaction of two or more agents so that their combined effect reduces the incidence of adverse event (AF) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35% and the adverse incidence rate is 20%, there is an improvement in the drug therapeutic regimen.

### Reduced Side Effects/Other Benefits

Combination therapies allow sympathomimetic agents in combination with anticonvulsant sulfamate compounds or an anticonvulsant sulfonylurea compounds to be administered in lower dosages to avoid side effects. In doing so, the concentration of each compound, required for mono-therapy, is reduced and the safe therapeutic range is extended.

### Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

What is claimed is:

1. A method of treating epilepsy comprising administering to a subject in need thereof an antiepileptic agent and a sympathomimetic agent.

2. The method of claim 1, wherein the antiepileptic agent is a sulfamate compound, or a sulfonylurea compound.

3. The method of claim 2, wherein the sulfamate compound is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

4. The method of claim 2, wherein the sulfonylurea is zonisamide.

5. The method of claim 1, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

6. The method of claim 3, wherein the topiramate is administered at between about 15 mg and about 1500 mg per day.

7. The method of claim 5, wherein the sympathomimetic agent is phentermine.

8. The method of claim 7, wherein the phentermine is administered at between about 2 mg and about 60 mg per day.

9. A method of treating diabetes comprising administering to a subject in need thereof an antiepileptic agent and a sympathomimetic agent.

10. The method of claim 9, wherein the antiepileptic agent is a sulfamate compound or a sulfonylurea compound.

11. The method of claim 10, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(l-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(i-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(l-methylethylidene)-4,5-O-sulfinyl-beta-D-fructopyranose sulfamate; (R)-2,3-O-(l-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

12. The method of claim 10, wherein the sulfonylurea is zonisamide.

13. The method of claim 9, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

14. The method of claim 11, wherein the topiramate is administered at between about 15 mg and about 1500 mg per day.

15. The method of claim 13, wherein the sympathomimetic agent is phentermine.

16. The method of claim 15, wherein the phentermine is administered at between about 2 mg and about 60 mg per day.

17. A method of treating hypertension or pulmonary hypertension comprising administering to a subject in need thereof an antiepileptic agent and a sympathomimetic agent.

18. The method of claim 17, wherein the antiepileptic agent is a sulfamate compound or a sulfonylurea compound.

19. The method of claim 18, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

20. The method of claim 17, wherein the sulfonylurea is zonisamide.

21. The method of claim 17, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

22. The method of claim 19, wherein the topiramate is administered at between about 15 mg and about 1500 mg per day.

23. The method of claim 21, wherein the sympathomimetic agent is phentermine.

24. The method of claim 23, wherein the phentermine is administered at between about 2 mg and about 60 mg per day.

25. A method of treating migraine comprising administering to a subject in need thereof an antiepileptic agent and a sympathomimetic agent.

26. The method of claim 25, wherein the antiepileptic agent is a sulfamate compound or a sulfonylurea compound.

27. The method of claim 26, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D)-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(l-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

28. The method of claim 25, wherein the sulfonylurea is zonisamide.

29. The method of claim 25, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

30. The method of claim 27, wherein the topiramate is administered at between about 20 mg and about 1500 mg per day.

31. The method of claim 29, wherein the sympathomimetic agent is phentermine.

32. The method of claim 31, wherein the phentermine is administered at between about 2 mg and about 60 mg per day.

33. A method of treating alcohol addiction comprising administering to a subject in need thereof an antiepileptic agent and a sympathomimetic agent.

34. The method of claim 33, wherein the antiepileptic agent is a sulfamate compound or a sulfonylurea compound.

35. The method of claim 34, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(l-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D)-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(l-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl-]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

36. The method of claim 34, wherein the sulfonylurea is zonisamide.

37. The method of claim 33, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

38. The method of claim 35, wherein the topiramate is administered at between about 20 mg and about 1500 mg per day.

39. The method of claim 37, wherein the sympathomimetic agent is phentermine.

40. The method of claim 39, wherein the phentermine is administered at between about 2 mg and about 60 mg per day.

41. A method of treating an impulse control disorder comprising administering to a subject in need thereof an antiepileptic agent and a sympathomimetic agent.

42. The method of claim 41, wherein the antiepileptic agent is a sulfamate compound or a sulfonylurea compound.

43. The method of claim 42, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethylidene)-beta-D-fructopyranose sulfamate.

44. The method of claim 42, wherein the sulfonylurea is zonisamide.

45. The method of claim 41, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

46. The method of claim 43, wherein the topiramate is administered at between about 15 mg and about 1500 mg per day.

47. The method of claim 45, wherein the sympathomimetic agent is phentermine.

48. The method of claim 47, wherein the phentermine is administered at between about 2 mg and about 60 mg per day.

49. A method of treating sleep apnea comprising administering to a subject in need thereof an antiepileptic agent and a sympathomimetic agent.

50. The method of claim 49, wherein the antiepileptic agent is a sulfamate compound or a sulfonylurea compound.

51. The method of claim 50, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(l-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,₅-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

52. The method of claim 50, wherein the sulfonylurea is zonisamide.

53. The method of claim 49, wherein the sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol., phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

54. The method of claim 51, wherein the topiramate is administered at between about 15 mg and about 1500 mg per day.

55. The method of claim 53, wherein the sympathomimetic agent is phentermine.

56. The method of claim 55, wherein the phentermine is administered at between about 2 mg and about 60 mg per day.

57. A pharmaceutical composition comprising an antiepileptic agent and bupropion.

58. The composition of claim 41, wherein the antiepileptic agent is a sulfamate compound or a sulfonylurea compound.

59. The composition of claim 42, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N-(4-methylbenzenesulfonyl)imidosulfonyl]-beta-D-fructopyranose sulfamate; 2,3-O-(1-methylethylidene)-4,₅-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(1-methylethy lidene)-beta-D-fructopyranose sulfamate.

60. The composition of claim 41, wherein the sulfonylurea is zonisamide.

61. The composition of claim 43, wherein the composition comprises between about 15 mg and about 1500 mg of topiramate.

62. A kit, comprising a therapeutically effective dose of an antiepileptic agent and bupropion, wherein the antiepileptic agent and the bupropion are in separate and discrete dosage forms, and instructions for its use.

63. The kit of claim 46, wherein the antiepileptic agent is a sulfamate compound or a sulfonylurea compound.

64. The kit of claim 47, wherein the sulfamate is selected from topiramate; 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(l-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O-[N -(4-methylbenzenesulfonyl)imidosulfonyl]-beta-D-fructopyranose sulfamate; 2,3-O-(l-methylethylidene)-4,5-O-[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate; and (S)-4,5-O-[N-(1,1-dimethylethoxycarbonyl)imidosulfinyl]-2,3-O-(l-methylethy lidene)-beta-D-fructopyranose sulfamate.

65. The kit of claim 47, wherein the sulfonylurea is zonisamide.

66. The kit of claim 46, wherein the composition comprises between about 20 mg and about 1500 mg of topiramate.

67. A method of ameliorating at least one side effect in a subject treated with a composition which possesses anorectic, sedative or central nervous system depressant properties by administering a therapeutically effective amount or a sub-therapeutic effective amount of a sympathomimetic agent such that at least one sedation or CNS side effect of said composition is inhibited or reduced.

68. The method of claim 67, wherein said composition is a centrally active agent, canabinoid antagonist or 5HT2C-selective serotonin receptor agonist.

69. The method of claim 68, wherein said centrally active agent is a sulfamate compound or a sulfonylurea compound.

70. The method of claim 68, wherein said canabinoid antagonist is rimonabant, MK-0364 or MK-0493.

71. The method of claim 68, wherein said 5HT2C-selective serotonin receptor agonist is locaserin HCl.

72. The method of claim 67, wherein said sympathomimetic agent is selected from bupropion, phenylethylamine, epinephrine, norepinephrine, epinine, dopamine, dobutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, propylhexedrine and diethylpropion.

73. The method of claim 72, wherein the sympathomimetic agent is phentermine.

74. The method of claim 73, wherein the phentermine is administered at between about 2 mg and about 60 mg per day.

75. A method of reducing at least one of the central nervous system side effects associated with monotherapeutic administration of a composition which possesses anorectic, sedative or central nervous system depressant properties in a subject so treated, the method comprising administering a combination of a sympathomimetic agent and said composition which possesses anorectic, sedative or central nervous system depressant properties, whereby the dose of the composition which possesses anorectic, sedative or central nervous system depressant properties in the combination is lower than that of the dose of the composition which possesses anorectic, sedative or central nervous system depressant properties in the monotherapy, wherein the monotherapy and the combination therapy produce similar therapeutic results.

76. A method of expanding the therapeutic window of a composition possessing anorectic, sedative, or central nervous system depressant properties, by administering a sympathomimetic agent in combination with said composition possessing anorectic, sedative, or central nervous system depressant properties.
